(19) United States
(12) Patent Application Publication
(10) Pub. No.: US 2025/0054625 A1
(43) Pub. Date: Feb. 13, 2025

(54) DETECTING ABNORMAL CELLS USING AUTOFLUORESCENCE MICROSCOPY

(71) Applicant: Verily Life Sciences LLC, South San Francisco, CA (US)

(72) Inventor: Carson McNeil, San Francisco, CA (US)

(73) Assignee: Verily Life Sciences LLC, South San Francisco, CA (US)

(21) Appl. No.: 18/719,561

(22) PCT Filed: Dec. 16, 2022
(86) PCT No.: PCT/US2022/081820

§ 371 Date: Jun. 13, 2024

Related U.S. Application Data
(60) Provisional application No. 63/295,261, filed on Dec. 30, 2021.

(51) Int. Cl. G16H 50/20; G06T 7/00; G06V 20/69

(52) U.S. Cl. CPC G16H 50/20; G06T 7/0014; G06V 20/69; G06T 2207/20081; G06T 2207/20084; G06T 2207/30024

(57) ABSTRACT

One example method includes receiving an image of a tissue sample stained with a stain; determining, by a first trained machine learning (“ML”) model using the image, a first set of abnormal cells in the tissue sample; receiving an autofluorescence image of the unstained tissue sample; determining, by a second trained ML model using the autofluorescence image and the first set of cells, a second set of abnormal cells, the second set of abnormal cells being a subset of the first set of abnormal cells; and identifying the abnormal cells of the second set of abnormal cells.

### FIELD

The present application generally relates to identifying abnormal cells in a tissue sample and more particularly relates to detecting abnormal cells using autofluorescence microscopy.

### BACKGROUND

Interpretation of tissue samples to determine the presence of cancer requires substantial training and experience with identifying features that may indicate cancer. Typically, a pathologist will receive a slide containing a slice of tissue and examine the tissue to identify features on the slide and determine whether those features likely indicate the presence of cancer, e.g., a tumor. In addition, the pathologist may also identify features, e.g., biomarkers, that may be used to diagnose a cancerous tumor, that may predict a risk for one or more types of cancer, or that may indicate a type of treatment that may be effective on a tumor.

### SUMMARY

Various examples are described for detecting abnormal cells using autofluorescence microscopy. One example method includes receiving an image of a tissue sample stained with a stain; determining, by a first trained machine learning (“ML”) model using the image, a first set of abnormal cells in the tissue sample; receiving an autofluorescence image of the unstained tissue sample; determining, by a second trained ML model using the autofluorescence image and the first set of cells, a second set of abnormal cells, the second set of abnormal cells being a subset of the first set of abnormal cells; and identifying the abnormal cells of the second set of abnormal cells.

One example system includes a non-transitory computer-readable medium; and one or more processors communicatively coupled to the non-transitory computer-readable medium, the one or more processors configured to execute processor-executable instructions stored in the non-transitory computer-readable medium to receive an image of a tissue sample stained with a stain; determine, by a first trained machine learning (“ML”) model using the image, a first set of abnormal cells in the tissue sample; receive an autofluorescence image of the unstained tissue sample; determine, by a second trained ML model using the autofluorescence image and the first set of cells, a second set of abnormal cells, the second set of abnormal cells being a subset of the first set of abnormal cells; and identify the abnormal cells of the second set of abnormal cells.

One example non-transitory computer-readable medium includes processor-executable instructions configured to cause one or more processors to receive an image of a tissue sample stained with a stain; determine, by a first trained machine learning (“ML”) model using the image, a first set of abnormal cells in the tissue sample; receive an autofluorescence image of the unstained tissue sample; determine, by a second trained ML model using the autofluorescence image and the first set of cells, a second set of abnormal cells, the second set of abnormal cells being a subset of the first set of abnormal cells; and identify the abnormal cells of the second set of abnormal cells.

These illustrative examples are mentioned not to limit or define the scope of this disclosure, but rather to provide examples to aid understanding thereof. Illustrative examples are discussed in the Detailed Description, which provides further description. Advantages offered by various examples may be further understood by examining this specification.

### BRIEF DESCRIPTION OF THE DRAWINGS

The accompanying drawings, which are incorporated into and constitute a part of this specification, illustrate one or more certain examples and, together with the description of the example, serve to explain the principles and implementations of the certain examples.

FIGS. 1-2 show example systems for detecting abnormal cells using autofluorescence microscopy;

FIG. 3 shows an example of abnormal cell analysis software for detecting abnormal cells using autofluorescence microscopy;

FIG. 4 shows an example graphical user interface for viewing visual indicators of abnormal cells;

FIG. 5 shows an example method for detecting abnormal cells using autofluorescence microscopy; and

FIG. 6 shows an example computing device suitable for use with various systems and methods for detecting abnormal cells using autofluorescence microscopy.

### DETAILED DESCRIPTION

Examples are described herein in the context of detecting abnormal cells using autofluorescence microscopy. Those of ordinary skill in the art will realize that the following description is illustrative only and is not intended to be in any way limiting. Reference will now be made in detail to implementations of examples as illustrated in the accompanying drawings. The same reference indicators will be used throughout the drawings and the following description to refer to the same or like items.

In the interest of clarity, not all of the routine features of the examples described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another.

To assist a pathologist in identifying abnormal cells in a tissue sample, the pathologist can capture an image of a slice of the tissue sample using an autofluorescence (“AF”) microscope. The AF image provides vectors of data indicating the magnitude of light captured at each of a number of wavelengths or wavelength ranges, rather than the red-green-blue (“RGB”) values from a conventional image sensor. Depending on the AF microscope, the vectors may have values for hundreds of different frequencies or frequency ranges corresponding to various compounds in the tissue, e.g., proteins, that are excited by laser light emitted by the AF microscope onto the tissue sample. Thus, while light is captured by the AF microscope, it does not necessarily provide an image that is easily interpretable by a human.

They can then stain the tissue sample using a suitable stain, such as a hematoxylin and eosin (“H&E”) stain, which may be applied virtually in some examples, and capture a second image using a conventional pathology microscope.

The image of the H&E-stained tissue sample is then presented to a trained ML model executed by a computing system, which identifies cells within the image and also identifies candidate abnormal cells, such as ballooning cells in the case of a tissue sample from a patient suspected of having non-alcoholic steatohepatitis (“NASH”), ductal carcinoma cells from a patient suspected of having breast cancer, or any cancerous cells in colorectal or other types of cancer. The computing system then receives the image from the AF microscope, aligns it with the image of the stained tissue, and identifies pixels within the AF image corresponding to identified abnormal cells in the image of the stained tissue. The system may perform some de-noising on the AF image and the performs a “max-pooling” operation whereby it selects, from all of the pixels for a specific abnormal cell, the maximum value for each frequency represented by the corresponding vectors. Thus, if a cell is represented by 100 pixels, the maximum value of each frequency (or frequency range) across each of the 100 pixels is used to construct a new vector containing those maximum values. However, for cells that are not identified as abnormal, no such vectors are created.

The max-pooled vectors are then input into a second trained ML model, which analyzes each of the inputted max-pooled vectors to determine whether any indicate an abnormal cell. For each abnormal cell that is determined from the max-pooled vectors, the corresponding candidate abnormal cell from the image of the H&E stained image is identified as being abnormal. For any candidate cells for which the second ML model does not determine it to be abnormal, the corresponding cell is indicated as being normal. Similarly, all of the cells not indicated as being abnormal by the first ML are indicated as normal.

The system can then output an indication of which cells in the image of the stained tissue are abnormal, such as by overlaying a visual indicator on those cells, e.g., text or a flag, or by shading or outlining the abnormal cells using a suitable color or pattern. The pathologist can then visually examine each of the identified abnormal cells to confirm or refute the determination from the system.

Such a system can provide much more rapid identification of abnormal cells within a tissue sample than a pathologist may otherwise be able to analyze. Further, by employing the cascade of two different ML models operating on two different types of images, the accuracy of the system can be significantly improved. In particular, an ML model can be trained to provide a very low false-negative rate, though at the expense of more false positives. By using a second microscope that analyzes laser-induced chromatic information from the same tissue sample, different features indicating an abnormality may be identified and used to confirm or refute the prediction from the first ML model. Thus, analysis of tissue samples can be made much more accurate, but with fewer false positives.

This illustrative example is given to introduce the reader to the general subject matter discussed herein and the disclosure is not limited to this example. The following sections describe various additional non-limiting examples and examples of detecting abnormal cells using autofluorescence microscopy.

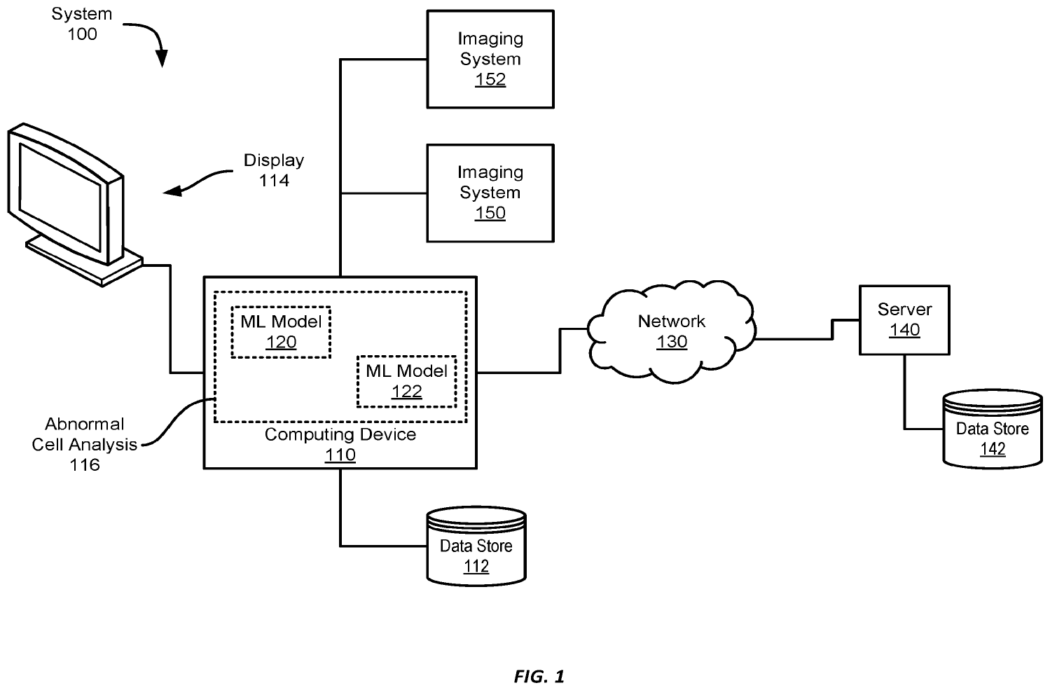

Referring now to FIG. 1, FIG. 1 shows an example system 100 for detecting abnormal cells using autofluorescence microscopy. The system 100 includes two imaging systems 150-152 that are connected to a computing device 110. The computing device 110 has abnormal cell analysis software 116, which includes two ML models 120-122, stored in memory and is connected to a display 114, a local data store 112, and to a remote server 140 via one or more communication networks 130. The remote server 140 is, in turn, connected to its own data store 142.

The imaging systems 150-152 each include a microscope and camera to capture images of pathology samples. Imaging system 150 in this example is a conventional pathology imaging system that captures digital images of tissue samples, stained or unstained, using broad-spectrum visible light. In contrast, imaging system 152 includes an AF microscope system which projects laser light onto tissue samples, which excites various molecules or compounds within the sample. The light emitted by the excited molecules or compounds is captured by the AF microscope system as a digital image having pixels with large numbers of frequency components.

The computing system 110 receives digital images from each of the imaging systems 150-152 corresponding to a particular tissue sample and provides them to the ML models 120-122 to identify one or more abnormal cells within the tissue sample.

In one scenario, a tissue sample will be prepared for imaging within the conventional imaging system 150, such as by obtaining a thin slice of tissue taken from a patient, staining it with a suitable stain (e.g., H&E), and positioning it on a slide, which is inserted into the imaging system 150. The imaging system 150 then captures an image of the stained sample (referred to as the “stained image”) and provides it to the computing device 110.

The stained tissue sample may then be washed of the stain and positioned on a slide, which is then inserted into the AF imaging system 152. The AF imaging system 152 captures an AF image of the unstained tissue sample and provides it to the computing device 110. Some workflows may involve capturing the AF image first before staining the tissue sample and imaging it with the conventional imaging system 150 because it may eliminate the step of washing the stain from the sample. But any suitable approach to capturing both images of the same tissue sample may be employed.

After receiving the captured stained image, the computing device 110 first executes ML model 120 to identify one or more candidate abnormal cells in the stained image. The computing device 110 then aligns the two images and determines pixels in the AF image corresponding to the candidate abnormal cells. After identifying those pixels in the AF image, it provides the AF image to the second ML model 122, such as by spatially collapsing candidate abnormal cells in the AF image and providing that collapsed data, which then determines whether each candidate abnormal cell is abnormal or not. The computing device 110 obtains the output from the second ML model 122 and generates indicators for each abnormal cell to identify the various abnormal cells within one or both images. It can then display one (or both) of the images on the display 114, along with the generated indicators, to enable a pathologist or other medical personnel to review the results.

And while in this example, the imaging systems 150-152 are connected to the computing device 110, such an arrangement is not needed. For example, an example system may omit one or both of the imaging systems 150-152 and the computing device 110 could instead obtain stained and AF images from its data store 112 or from the remote server 140. Similarly, while the abnormal cell analysis is performed at the computing device 110, in some examples, stained and AF images may be provided to the remote server 140, which may execute abnormal cell analysis software 116, including suitable ML models, e.g., ML models 120-122.

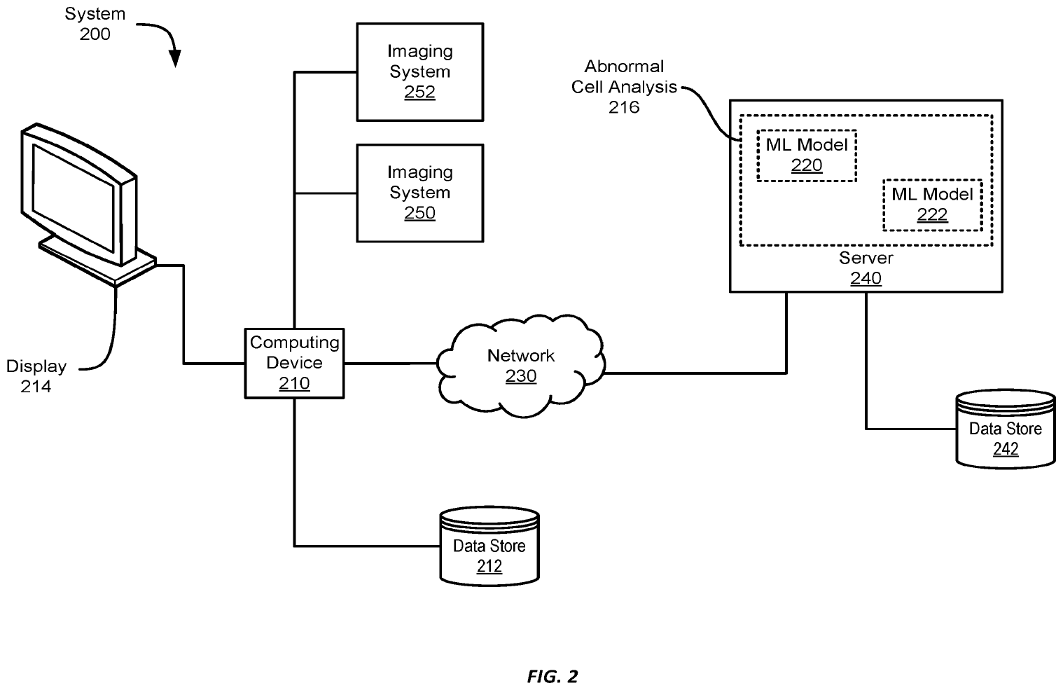

Referring now to FIG. 2, FIG. 2 shows another example system for detecting abnormal cells using AF microscopy. In this example, the system includes components similar to those shown in the system 100 of FIG. 1. In particular, the system 200 includes a computing device 210 with a display 214 and a local data store 212. Two imaging systems 250-252 are connected to the computing device 210. The computing device 210 is connected to a remote server 240 via one or more communication networks 240. The remote server 240 in this example includes abnormal cell analysis software 212, which includes two ML models 220-222, stored in memory.

In operation, the computing device 210 receives stained and AF images from the imaging systems 250-252 or the data store 212. It then provides those images to the server 240, which executes the abnormal cell analysis software 212 to identify one or more abnormal cells using the two ML models 220-222. It then provides the results of the analysis to the computing device 210, which can display any identified abnormal cells on the display 214.

Such an example system 200 may provide advantages in that it may allow a medical center to invest in imaging equipment, but employs a service provider to analyze captured images, rather than requiring the medical center to perform its own analysis. This can enable smaller medical centers, or medical centers serving remote populations, to provide high quality diagnostic services without requiring them to take on the expense of performing its own analysis.

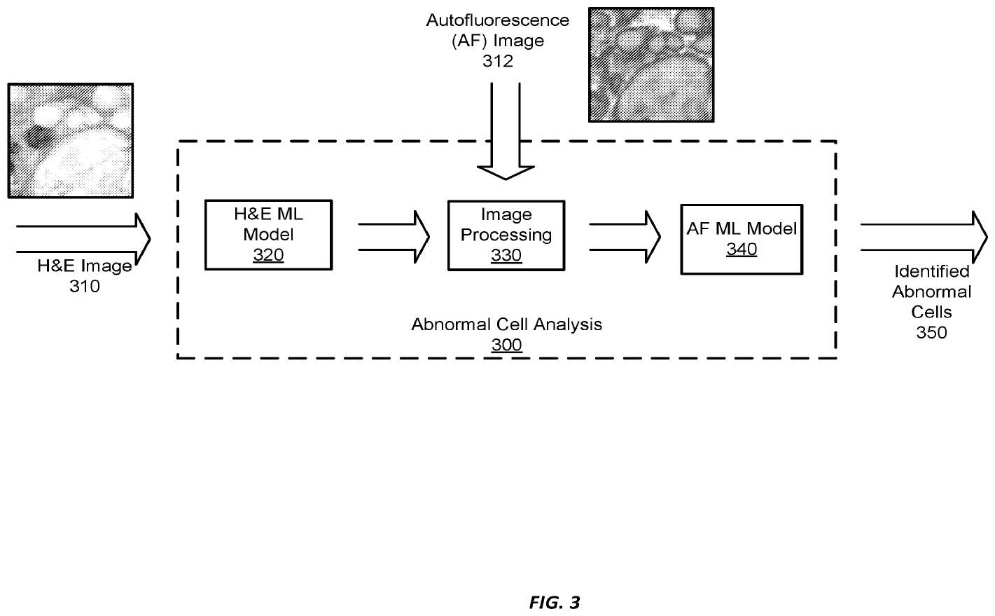

Referring now to FIG. 3, FIG. 3 shows a block diagram of example abnormal cell analysis software 300 (or “analysis software 300”). The analysis software 300 includes two trained ML models: an H&E ML model 320 and an AF ML model 340. Each of the ML models 320, 340 have been trained on respective training data sets to identify abnormal cells in stained images, in the case of H&E ML model 320, or to identify abnormal cells in AF images, in the case of AM ML model 340. In addition, the analysis software 300 includes image processing functionality 330 that receives output from the H&E ML model 320 and corresponding AF images 312. And while ML model 320 in this example is an H&E-trained ML model, any suitable stains may be used, such as trichrome or any immunohistochemistry stains.

As discussed above with respect to FIGS. 1-2, stained and AF images of tissue samples may be captured by respective imaging systems, e.g., imaging systems 150-152, 250-252, and provided to a computing device executing abnormal cell analysis software. In this example, the analysis software 300 is executed by any suitable computing device, such as computing devices 120, 220 or remote servers 140, 240. The analysis software 300 receives H&E-stained images 310 and AF images 312. The images 310, 312 may be received from a corresponding imaging system, from a local data store, or from a remote computing device, as illustrated in FIGS. 1-2.

After receiving an H&E image, the trained H&E ML model identifies one or more candidate abnormal cells in the H&E image. In this example, the H&E ML model is a neural network, e.g., Inception V3 from GOOGLE LLC; however, any suitable type of ML model may be used, such as a deep convolutional neural network, a residual neural network (“Resnet”) or NASNET provided by GOOGLE LLC from MOUNTAIN VIEW, CALIFORNIA, or a recurrent neural network, e.g. long short-term memory (“LSTM”) models or gated recurrent units (“GRUs”) models. The ML models 212, 222 can also be any other suitable ML model, such as a three-dimensional CNN (“3DCNN”), a dynamic time warping (“DTW”) technique, a hidden Markov model (“HMM”), etc., or combinations of one or more of such techniques—e.g., CNN-HMM or MCNN (Multi-Scale Convolutional Neural Network). Further, some examples may employ adversarial networks, such as generative adversarial networks (“GANs”), or may employ autoencoders (“AEs”) in conjunction with ML models, such as AEGANs or variational AEGANs (“VAEGANs”).

The H&E ML model identifies individual cells within the H&E image and identifies candidate abnormal cells. In this disclosure, the output of the H&E ML model is a “candidate” abnormal cell because the AF ML model 340 makes the ultimate determination as to whether a particular cell is abnormal. Absent the use of the AF ML model 340, the output of the H&E ML model may be considered as the set of abnormal cells and annotated as such for display on a display device. However, in this example analysis software 300, the H&E ML model has been trained and tuned to be overinclusive in identifying cells as abnormal, thus it may have a higher than desirable false-positive rate, if it were used as a standalone ML model. However, the training and tuning has been performed, in this example, such that the false-negative rate is exceedingly low, i.e., approaching zero. This may enable the AF ML model 340 to operate on only true-positive or false-positive candidate abnormal cells without concern that false-negatives may escape detection.

The H&E ML model 320 outputs information identifying individual cells identified in the H&E image and which of those cells is identified as being abnormal. Such information may be used to identify pixels within a corresponding AF image that are associated with identified abnormal cells.

The image processing 330 component in this example performs the mapping from candidate abnormal cells identified in the H&E image 310 to corresponding pixels within the AF image 312. This process may involve using conventional alignment and warping functionality on the H&E and AF images to align the two images to enable identifying pixels in the AF image corresponding to the candidate abnormal cells in the H&E image.

Once the images are aligned, the image processing component 330 may provide the AF image and information identifying the relevant pixels to the AF ML model 340. Such information may include identifying all pixels corresponding to each candidate abnormal cell, boundaries of pixels in the AF image corresponding to each candidate abnormal cell, etc.

In this example, the image processing component 330 identifies all pixels corresponding to each candidate abnormal cell, and for each candidate abnormal cell, performs a “max-pooling” operation to generate a single “pixel value” for the cell.

As discussed above, each pixel in an AF image may include a large vector of channel values. Depending on the AF imaging device used, the number of channels per pixel may be in the hundreds, each representing a particular frequency or frequencies, which is far more channels per pixel than a typical visible light image that employs three color channels: red, green, and blue. Thus, to perform max-pooling, the image processing component 330 analyzes each frequency channel for each pixel of a particular candidate abnormal cell in the AF image and identifies the maximum value for that frequency channel among those pixels. It then constructs a new “pixel” having the maximum values for each frequency channel to represent the candidate abnormal cell. Such an operation collapses the spatial dimensions of the candidate abnormal cell and reduces it to a single pixel. It then provides the collapsed pixels for a particular candidate abnormal cell as an input vector for the candidate abnormal cells to the AF ML model 340, which determines whether each input vector represents an abnormal cell. And while this example employs a max-pooling approach, other methods of collapsing spatial dimensions may be employed. For example, the image processing component 330 may average one or more frequency channels across each pixel within a candidate abnormal cell to generate an input vector for the candidate abnormal cell.

After receiving the input vectors from the image processing component 330, the AF ML model identifies which of the candidate abnormal cells is a true-positive and which is a false-positive and outputs indications of the true-positive abnormal cells. In this example, the AF ML model is a trained support vector machine (“SVM”); however, as with the H&E ML model 320, any suitable type of ML model may be employed, such as those discussed above. Thus, for each candidate abnormal cell that the AF ML model 340 identifies as an abnormal cell, the candidate abnormal cell is identified as a true-positive abnormal cell, while the remaining candidate abnormal cells are identified as false-positive abnormal cells.

The identified true-positive abnormal cells 350 are then identified within either (or both) of the H&E or AF images, such as by tagging the corresponding location within the image, identifying a cell number within the image, etc. The identified true-positive abnormal cells 350 may then be displayed on a display 114 or transmitted to a remote computing device. For example, if the computing device 210 in FIG. 2 provides H&E and AF images to the remote server 240, the remote server 240 may then provide the identified true-positive abnormal cells 350 to the computing device 210.

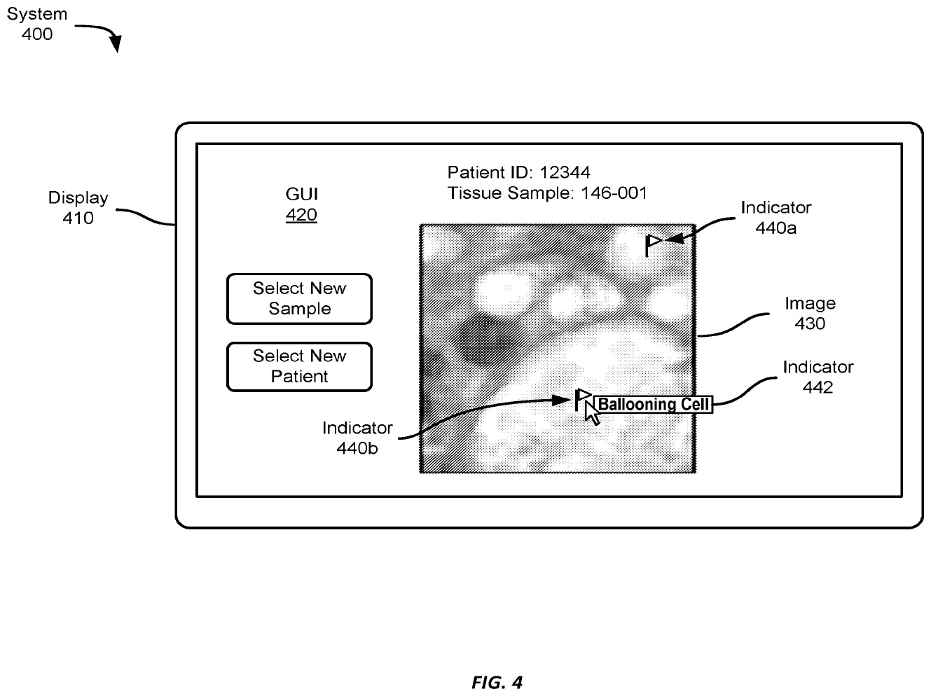

FIG. 4 illustrates an example system for displaying indicators of abnormal cells identified by the example analysis software 300 in FIG. 3. In this example, the system 400 includes a display 410 that displays a graphical user interface (“GUI”). The GUI 420 displays an image 430 that includes one or both of the AF or H&E images 310, 312 (or a portion of an image) inputted into the analysis software 300. It then applies visual indicators 440a-b to each abnormal cell visible in the image. In this example, the system 400 displays a flag indicator 440a-b on each visible, identified abnormal cell. Further, the user may interact with the visual indicator to obtain additional information about the cell. In this example, the image is of liver tissue from a patient with suspected nonalcoholic steatohepatitis. When the user moves a mouse cursor in proximity to the visual indicator 440b, the type of abnormal cell is displayed as a separate indicator 442, which identifies the abnormal cell as a ballooning cell in this example. In some examples additional information may be provided, such as a severity level associated with the abnormal cell. Still any other suitable information may be displayed as well. And while flags and text overlays are depicted in this example, any suitable visual indicator may be employed according to different examples, such as by coloring, shading, or outlining the identified abnormal cells.

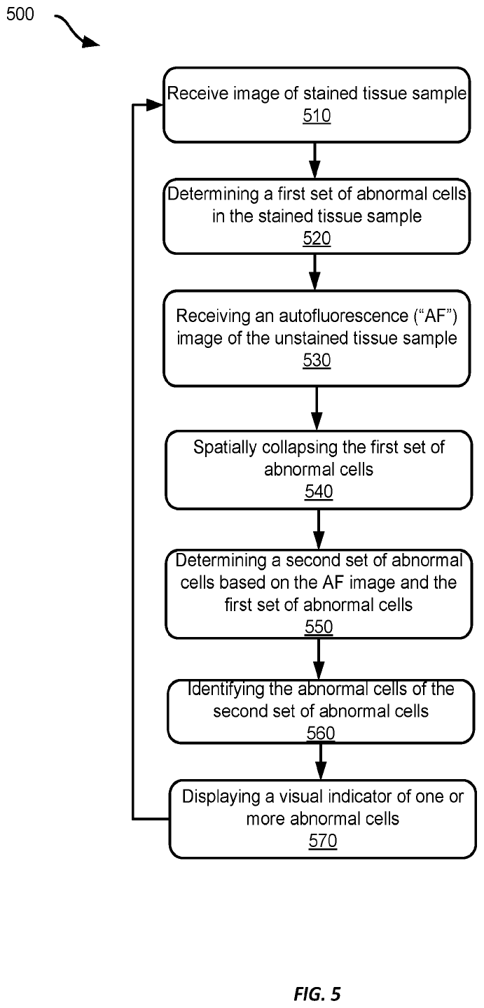

Referring now to FIG. 5, FIG. 5 shows an example method 500 for detecting abnormal cells using AF microscopy. The method 500 of FIG. 5 will be discussed with respect to the example system 100 shown in FIGS. 1 and 3; however, any system according to this disclosure may be employed, including the example system 200 shown in FIG. 2.

At block 510, the computing device 110 receives an image of a tissue sample stained with a stain (also referred to as a “stained image”). In this example, the computing device 110 receives the stained image from imaging system 150 and provides it to the analysis software 116, 300. In some examples however, the computing device 110 may receive the stained image from another source. For example, the data store 112 may have one or more stained images from which the analysis software 116, 300 can access and receive a stained image. Further, in some examples, the computing device 112 may receive stained images from a remote computing device, such as server 140, which may have one or more stained images stored in its data store 142.

In some examples, a cloud-style configuration may be employed, similar to the example system 200 in FIG. 2. In such an example, the remote server 240 may receive a stained image from computing device 210. The stained image may have been captured by imaging system 250 or retrieved from datastore 212 and then provided to the remote server 240 via the network. Alternatively, stained images may be provided to the remote server 240 from the computing device 210 and then stored in the data store 242. At a later time, the remote server 240 may execute analysis software 216, which receives the stained image from the data store 242. Still further techniques may be employed in some examples. For example, a medical services provider may store images captured by imaging systems in cloud storage, which may then be accessed by analysis software executed locally at the medical services provider, e.g., by a computing device 110, or by analysis software executed remotely, e.g., by a service provider operating remote server 240. In some such examples, the analysis software 116 may receive the stained image from cloud storage.

At block 520, the analysis software 116, 300 uses a trained ML model to determine a first set of abnormal cells in the stained image. As discussed above with respect to FIG. 3, the analysis software 300 employs a trained ML model to identify one or more candidate abnormal cells in the stained image. While the example analysis software employs a ML model 320 trained to analyze H&E-stained images, any suitable stain may be employed that corresponds with the trained ML model used by the analysis software. After providing the stained image to the trained ML model 320, the ML model 320 outputs identifications of any candidate abnormal cells identified in the stained image. It may output additional information as well, such as all identified cells within the image, whether abnormal or otherwise, locations of cells within the stained image, boundaries of cells within stained image (e.g., cell membranes), or identifiers for cells within the stained image.

At block 530, the analysis software 116, 300 receives an AF image of the unstained tissue sample. The AF image may be received in any manner according to this disclosure, such as described above with respect to block 510.

At block 540, the analysis software 116, 300 employs an image processing component 330 to spatially collapse the first set of abnormal cells into corresponding input vectors. For example, as discussed above with respect to FIG. 3, the image processing component 330 may align the AF image with the stained image, and then identify pixels in the AF image that correspond to the first set of abnormal cells, which include candidate abnormal cells identified by the first ML model 320.

After identifying pixels in the AF image that correspond to the first set of abnormal cells, the image processing component 330 performs a max-pooling operation for each cell in the first set of abnormal cells using the corresponding pixels in the AF image. Thus, for each pixel in the AF image corresponding to a particular candidate abnormal cell, the image processing component 330 identifies the maximum value for each frequency channel and stores it in a corresponding location in an input vector for the candidate abnormal cell. Once a maximum value for each frequency channel has been identified and inserted into the input vector, the image processing component 330 performs the same operations for any remaining candidate abnormal cells to create a set of input vectors.

While this example employs max-pooling to collapse the spatial dimensions of each candidate abnormal cell in the AF image, other approaches may be employed instead. For example, and as discussed above with respect to FIG. 3, the image processing component 330 may average each frequency channel and store the average values in an input vector. In some examples, the image processing component 330 may only average frequency channels having a value satisfying a predefined threshold or may input a minimum value for frequency channels where no pixel has a corresponding frequency value satisfying a predefined threshold. Still other approaches to collapsing the spatial dimensions of candidate abnormal cells in the AF image may be employed. Further, it should be appreciated that block 540 may be optional in some examples. Instead, the candidate abnormal cells from the AF image may be analyzed without first being spatially collapsed.

At block 550, the analysis software 116, 300 uses a second trained ML model to determine a second set of abnormal cells based on the AF image and the first set of abnormal cells. As discussed above, such as with respect to FIG. 3, the trained AF ML model 340 receives information obtained from the AF image, such as spatially collapsed input vectors or pixels corresponding to candidate abnormal cells, and determines one or more abnormal cells based on such information. In this example, the AF ML model 340 was trained using spatially collapsed input vectors, and thus it obtains one or more input vectors from the image processing component 340 corresponding to the abnormal cells in the first set of abnormal cells identified by the first ML model 320. However, depending on how the AF ML model 340 was trained, different types of input information may be employed, as discussed above.

At block 560, the analysis software identifies the cells in the second set of abnormal cells as the abnormal cells within the tissue sample. For any cells that are in the first set of abnormal cells but not in the second set of abnormal cells, the analysis software 116, 300 identifies them as normal cells.

At block 570, the computing device 110 displays one or more visual indicators identifying corresponding abnormal cells within one of the stained image or the AF image. For example, as illustrated in FIG. 4, the computing device 110 may display the stained or AF image 430 on its display 114 as well as visual indicators 420a-b overlaid on the image 430 to identify abnormal cells. Other kinds of visual indicators may be provided, such as context-sensitive indicators, such as indicator 442, which appears when a user positions a mouse cursor near a displayed visual indicator. However, other types of visual indicators may be employed. For example, the computing device 110 may apply shading or a colored overlay on identified abnormal cells displayed in the image 430. For example, abnormal cells may be shaded red, while normal cells are unshaded or shaded a different color. Alternatively, abnormal cells may be outlined using a predefined color to identify them. In some examples, where abnormal cells have different levels of severity associated with them, a visual indicator may indicate a level of severity, such as by using different colors for different severities or by displaying a severity in response to a user interacting with the abnormal cell, e.g., by touching it on a touch-sensitive display or moving a cursor over the abnormal cell. Still other kinds of indicators may be employed according to different examples.

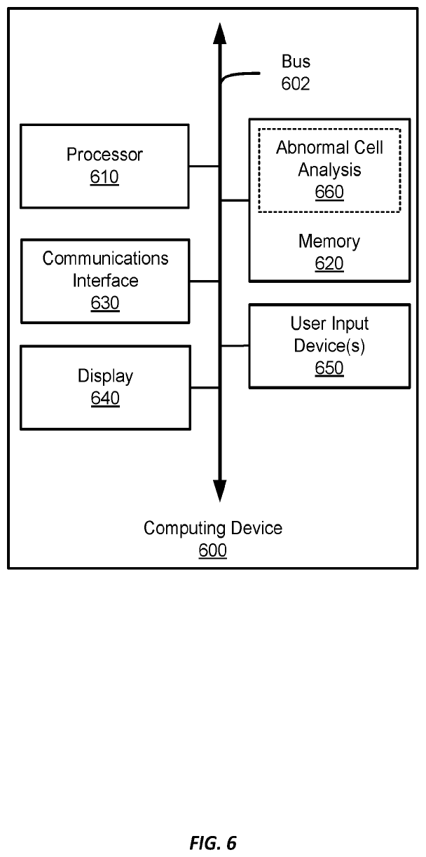

Referring now to FIG. 6, FIG. 6 shows an example computing device 600 suitable for use in example systems or methods for detecting abnormal cells using AF microscopy according to this disclosure. The example computing device 600 includes a processor 610 which is in communication with the memory 620 and other components of the computing device 600 using one or more communications buses 602. The processor 610 is configured to execute processor-executable instructions stored in the memory 620 to perform one or more methods for detecting abnormal cells using AF microscopy according to different examples, such as part or all of the example method 500 described above with respect to FIG. 5. In this example, the memory 620 includes abnormal cell analysis software 660, such discussed above with respect to FIG. 3. In addition, the computing device 600 also includes one or more user input devices 650, such as a keyboard, mouse, touchscreen, microphone, etc., to accept user input; however, in some examples, the computing device 600 may lack such user input devices, such as remote servers or cloud servers. The computing device 600 also includes a display 640 to provide visual output to a user.

The computing device 600 also includes a communications interface 640. In some examples, the communications interface 630 may enable communications using one or more networks, including a local area network (“LAN”); wide area network (“WAN”), such as the Internet; metropolitan area network (“MAN”); point-to-point or peer-to-peer connection; etc. Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

While some examples of methods and systems herein are described in terms of software executing on various machines, example methods and systems may also be implemented as specifically-configured hardware, such as field-programmable gate array (FPGA) specifically to execute the various methods according to this disclosure. For example, examples can be implemented in digital electronic circuitry, or in computer hardware, firmware, software, or in a combination thereof. In one example, a device may include a processor or processors. The processor comprises a computer-readable medium, such as a random access memory (RAM) coupled to the processor. The processor executes computer-executable program instructions stored in memory, such as executing one or more computer programs. Such processors may comprise a microprocessor, a digital signal processor (DSP), an application-specific integrated circuit (ASIC), field programmable gate arrays (FPGAs), and state machines. Such processors may further comprise programmable electronic devices such as PLCs, programmable interrupt controllers (PICs), programmable logic devices (PLDs), programmable read-only memories (PROMs), electronically programmable read-only memories (EPROMs or EEPROMs), or other similar devices.

Such processors may comprise, or may be in communication with, media, for example one or more non-transitory computer-readable media, that may store processor-executable instructions that, when executed by the processor, can cause the processor to perform methods according to this disclosure as carried out, or assisted, by a processor. Examples of non-transitory computer-readable medium may include, but are not limited to, an electronic, optical, magnetic, or other storage device capable of providing a processor, such as the processor in a web server, with processor-executable instructions. Other examples of non-transitory computer-readable media include, but are not limited to, a floppy disk, CD-ROM, magnetic disk, memory chip, ROM, RAM, ASIC, configured processor, all optical media, all magnetic tape or other magnetic media, or any other medium from which a computer processor can read. The processor, and the processing, described may be in one or more structures, and may be dispersed through one or more structures. The processor may comprise code to carry out methods (or parts of methods) according to this disclosure.

The foregoing description of some examples has been presented only for the purpose of illustration and description and is not intended to be exhaustive or to limit the disclosure to the precise forms disclosed. Numerous modifications and adaptations thereof will be apparent to those skilled in the art without departing from the spirit and scope of the disclosure.

Reference herein to an example or implementation means that a particular feature, structure, operation, or other characteristic described in connection with the example may be included in at least one implementation of the disclosure. The disclosure is not restricted to the particular examples or implementations described as such. The appearance of the phrases “in one example,” “in an example,” “in one implementation,” or “in an implementation,” or variations of the same in various places in the specification does not necessarily refer to the same example or implementation. Any particular feature, structure, operation, or other characteristic described in this specification in relation to one example or implementation may be combined with other features, structures, operations, or other characteristics described in respect of any other example or implementation.

Use herein of the word “or” is intended to cover inclusive and exclusive OR conditions. In other words, A or B or C includes any or all of the following alternative combinations as appropriate for a particular usage: A alone; B alone; C alone; A and B only; A and C only; B and C only; and A and B and C.

1. A method comprising:
receiving an image of a tissue sample stained with a stain;
determining, by a first trained machine learning (“ML”) model using the image, a first set of abnormal cells in the tissue sample;
receiving an autofluorescence image of the unstained tissue sample;
determining, by a second trained ML model using the autofluorescence image and the first set of cells, a second set of abnormal cells, the second set of abnormal cells being a subset of the first set of abnormal cells; and
identifying the abnormal cells of the second set of abnormal cells.

2. The method of claim 1, wherein the autofluorescence image comprises a plurality of pixels and a vector of frequency channels per pixel, and further comprising:
for each abnormal cell in the first set of abnormal cells:
determining a set of pixels corresponding to the respective abnormal cell, and
generating an input vector from the vectors of the frequency channels for the set of pixels; and
wherein determining the second set of abnormal cells is based on the generated input vectors.

3. The method of claim 2, wherein generating the input vector for each abnormal cell comprises:
determining a maximum value for each frequency channel within the set of pixels, and
generating the input vector comprising, for each color channel, the maximum value of the respective color channel.

4. The method of claim 2, wherein generating the input vector for each abnormal cell comprises:
determining an average value for each frequency channel within the set of pixels, and
generating the input vector comprising, for each color channel, the average value of the respective frequency channel.

5. The method of claim 1, wherein identifying the abnormal cells comprises providing a visual indicator on the image of a tissue sample.

6. The method of claim 1, wherein the stain comprises a virtual stain.

7. The method of claim 1, wherein the stain comprises a hematoxylin and eosin (“H&E”) stain.

8. The method of claim 1, wherein the abnormal cells are ballooning cells associated with nonalcoholic steatohepatitis.

9. A system comprising:
a non-transitory computer-readable medium; and
one or more processors communicatively coupled to the non-transitory computer-readable medium, the one or more processors configured to execute processor-executable instructions stored in the non-transitory computer-readable medium to:
receive an image of a tissue sample stained with a stain;
determine, by a first trained machine learning (“ML”) model using the image, a first set of abnormal cells in the tissue sample;
receive an autofluorescence image of the unstained tissue sample;
determine, by a second trained ML model using the autofluorescence image and the first set of cells, a second set of abnormal cells, the second set of abnormal cells being a subset of the first set of abnormal cells; and
identify the abnormal cells of the second set of abnormal cells.

10. The system of claim 9, wherein the autofluorescence image comprises a plurality of pixels and a vector of frequency channels per pixel, and wherein the one or more processors configured to execute further processor-executable instructions stored in the non-transitory computer-readable medium to, for each abnormal cell in the first set of abnormal cells:
determine a set of pixels corresponding to the respective abnormal cell, and
generate an input vector from the vectors of the frequency channels for the set of pixels; and
determine, by the second trained ML model using the autofluorescence image and the first set of cells, including the input vectors, the second set of abnormal cells.

11. The system of claim 10, wherein the one or more processors configured to execute further processor-executable instructions stored in the non-transitory computer-readable medium to:
determine a maximum value for each frequency channel within the set of pixels, and
generate the input vector comprising, for each color channel, the maximum value of the respective color channel.

12. The system of claim 10, wherein the one or more processors configured to execute further processor-executable instructions stored in the non-transitory computer-readable medium to:
determine an average value for each frequency channel within the set of pixels, and
generate the input vector comprising, for each color channel, the average value of the respective frequency channel.

13. The system of claim 9, wherein the one or more processors configured to execute further processor-executable instructions stored in the non-transitory computer-readable medium to provide a visual indicator on the image of a tissue sample.

14. (canceled)

15. (canceled)

16. The system of claim 9, wherein the abnormal cells are ballooning cells associated with nonalcoholic steatohepatitis.

17. A non-transitory computer-readable medium comprising processor-executable instructions configured to cause one or more processors to:
receive an image of a tissue sample stained with a stain;
determine, by a first trained machine learning (“ML”) model using the image, a first set of abnormal cells in the tissue sample;
receive an autofluorescence image of the unstained tissue sample;
determine, by a second trained ML model using the autofluorescence image and the first set of cells, a second set of abnormal cells, the second set of abnormal cells being a subset of the first set of abnormal cells; and
identify the abnormal cells of the second set of abnormal cells.

18. The non-transitory computer-readable medium of claim 17, wherein the autofluorescence image comprises a plurality of pixels and a vector of frequency channels per pixel, and further comprising processor-executable instructions configured to cause the one or more processors to, for each abnormal cell in the first set of abnormal cells:
determine a set of pixels corresponding to the respective abnormal cell, and
generate an input vector from the vectors of the frequency channels for the set of pixels; and
determine, by the second trained ML model using the autofluorescence image and the first set of cells, including the input vectors, the second set of abnormal cells.

19. The non-transitory computer-readable medium of claim 18, further comprising processor-executable instructions configured to cause the one or more processors to:
determine a maximum value for each frequency channel within the set of pixels, and
generate the input vector comprising, for each color channel, the maximum value of the respective color channel.

20. The non-transitory computer-readable medium of claim 18, further comprising processor-executable instructions configured to cause the one or more processors to:
determine an average value for each frequency channel within the set of pixels, and
generate the input vector comprising, for each color channel, the average value of the respective frequency channel.

21. The non-transitory computer-readable medium of claim 17, further comprising processor-executable instructions configured to cause the one or more processors to provide a visual indicator on the image of a tissue sample.

22. (canceled)

23. (canceled)

24. The system of claim 9, wherein the abnormal cells are ballooning cells associated with nonalcoholic steatohepatitis.
